(19) United States
(12) Patent Application Publication
(10) Pub. No.: US 2025/0009248 A1
(43) Pub. Date: Jan. 9, 2025

(54) ENDOSCOPY SUPPORT SYSTEM, ENDOSCOPY SUPPORT METHOD, AND STORAGE MEDIUM

(71) Applicant: OLYMPUS MEDICAL SYSTEMS CORP., Tokyo (JP)

(72) Inventors: Jun HANE, Tokyo (JP); Hiromasa FUJITA, Tokyo (JP); Kensuke MIYAKE, Tokyo (JP)

(73) Assignee: OLYMPUS MEDICAL SYSTEMS CORP., Tokyo (JP)

(21) Appl. No.: 18/892,693

(22) Filed: Sep. 23, 2024

Related U.S. Application Data
(63) Continuation of application No. PCT/JP2022/013531, filed on Mar. 23, 2022.

(51) Int. Cl. A61B 5/06; A61B 1/00; A61B 1/31; A61B 34/20; A61M 25/01

(52) U.S. Cl. CPC A61B 5/065; A61B 1/0005; A61B 1/31; A61B 34/20; A61B 2034/2061; A61B 2034/2065; A61M 2025/0166

(57) ABSTRACT

In endoscopy, each of a display controller and a recording controller acquires a first curved shape of an endoscope insertion portion when an endoscope distal end portion is located at a predetermined site, an endoscopic image captured in the endoscopy, and a second shape of an endoscope insertion portion when the endoscopic image is captured. The recording controller may record the first curved shape of the endoscope insertion portion, the endoscopic image, and the second curved shape of the endoscope insertion portion in a storage device. The display controller may simultaneously display the first curved shape of the endoscope insertion portion and the second curved shape of the endoscope insertion portion on a display device.

### CROSS-REFERENCE TO RELATED APPLICATIONS

This application is based upon and claims the benefit of priority from the International Application No. PCT/JP2022/013531, filed on Mar. 23, 2022, the entire contents of which are incorporated.

### BACKGROUND

### 1. Technical Field

The present disclosure relates to an endoscopy support system, an endoscopy support method, and a storage medium for supporting an operator in endoscopy.

### 2. Description of the Related Art

In colonoscopy, in order to facilitate treatment to be performed at a later date and follow-up after the treatment, when a lesion (for example, polyp or cancer) is confirmed, it is common to record where the confirmed lesion is. In relation to this, a method has been proposed in which a lesion detected by AI is marked at a corresponding part in a large intestine diagram at the time of colonoscopy.

### SUMMARY

An endoscopy support system according to an aspect of the present disclosure includes one or more processors having hardware. The processor is structured to acquire, in endoscopy, a first curved shape of an endoscope insertion portion when an endoscope distal end portion is located at a predetermined site, an endoscopic image captured in the endoscopy, and a second curved shape of an endoscope insertion portion when the endoscopic image is captured, and the processor is structured to simultaneously display the first curved shape of the endoscope insertion portion and the second curved shape of the endoscope insertion portion on a monitor.

Another aspect of the present disclosure is an endoscopy support method. The endoscopy support method includes acquiring, in endoscopy, a first curved shape of an endoscope insertion portion when an endoscope distal end portion is located at a predetermined site, an endoscopic image captured in the endoscopy, and a second curved shape of an endoscope insertion portion when the endoscopic image is captured, and simultaneously displaying the first curved shape of the endoscope insertion portion and the second curved shape of the endoscope insertion portion on a monitor.

Note that any combination of the above components and modifications of the expressions of the present disclosure among methods, devices, systems, recording media, computer programs, and the like are also effective as aspects of the present disclosure.

### BRIEF DESCRIPTION OF THE DRAWINGS

FIG. 1 is a diagram illustrating an overall system configuration related to colonoscopy according to an embodiment;

FIG. 2 is a diagram illustrating an example of an endoscope used in the present embodiment;

FIG. 3 is a diagram illustrating a configuration example of an endoscopy support system according to the embodiment;

FIG. 4 is a diagram illustrating a first screen example displayed on a display device;

FIG. 5 is a diagram illustrating a second screen example displayed on the display device;

FIG. 6 is a diagram illustrating a third screen example displayed on the display device;

FIG. 7 is a diagram illustrating a fourth screen example displayed on the display device;

FIG. 8 is a diagram illustrating a fifth screen example displayed on the display device;

FIG. 9 is a diagram illustrating a sixth screen example displayed on the display device;

FIGS. 10A to 10C are diagrams illustrating examples in which a plurality of endoscope shapes are displayed as trigonometric projections in three directions;

FIG. 11 is a diagram illustrating an example in which a three-dimensional endoscope shape at the time of reaching the cecum is displayed in a bird's eye perspective view;

FIG. 12 is a flowchart illustrating an operation of the endoscopy support system according to the embodiment at the end of an examination; and

FIG. 13 is a flowchart illustrating an operation of the endoscopy support system according to the embodiment at the time of confirming examination information.

### DETAILED DESCRIPTION

The invention will now be described by reference to the preferred embodiments. This does not intend to limit the scope of the present invention, but to exemplify the invention.

The present embodiment relates to colonoscopy. In colonoscopy, an endoscope is inserted into the cecum, and at the time of removal, lesion screening, lesion examination, and lesion treatment are performed. When a lesion is confirmed, where the lesion is recorded, in order to facilitate treatment to be performed at a later date and follow-up after the treatment. In addition, as a result of the examination, even in a case where treatment is not performed for the reason of not being a lesion or the like, follow-up may be performed to determine whether the portion is diseased, and the position of the portion may be recorded as a lesion candidate.

Reaccessing a lesion or lesion candidate found in a previous examination is routinely performed, such as when treatment is required in a pathology diagnosis after lesion screening in colonoscopy. The same doctor may reaccess or another doctor may reaccess. In addition, there is a case where reaccess is made at another facility.

However, the large intestine has a length of about 1 to 1.5 m and is easily deformed. In particular, the insertion length from the anus to the same lesion largely changes depending on the insertion method at the time of insertion. It may not be easily reaccessible by the same doctor. Reaccess may be made at other facilities from the viewpoint of expertise, but reaccess by other doctors is more difficult. Although a schema diagram of the large intestine and an approximate lesion position may be described in a referral, it is often the case that the lesion is not found even if reaccess is made with reference to the schema diagram. Therefore, information related to the lesion position exchanged between doctors and facilities is for reference only. From the above, it is required to indicate a lesion position or a lesion candidate position with good reproducibility.

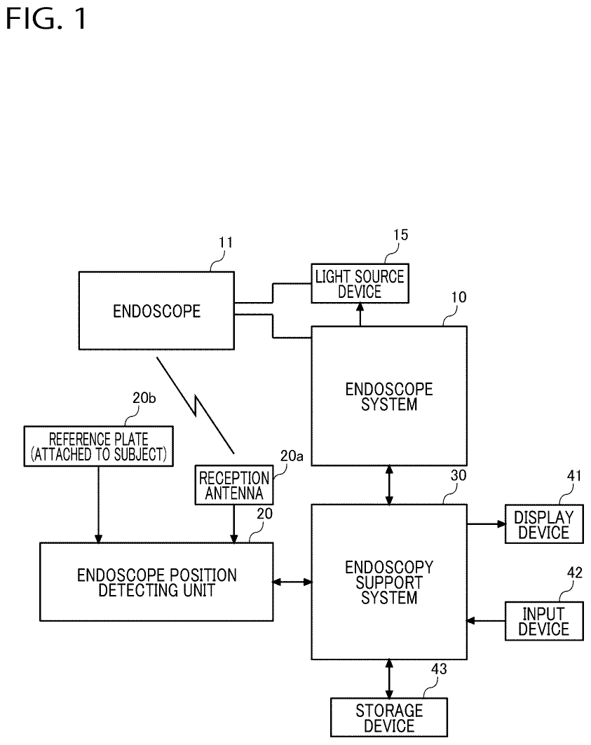

FIG. 1 is a diagram illustrating an overall system configuration related to colonoscopy according to an embodiment. In the present embodiment, an endoscope system 10, an endoscope 11, a light source device 15, an endoscope position detecting unit (UPD) 20, an endoscopy support system 30, a display device 41, an input device 42, and a storage device 43 are used. The endoscope 11 according to the present embodiment is a large intestine endoscope to be inserted into the large intestine of a subject (patient).

The endoscope 11 includes a lens and a solid-state imaging element (for example, CMOS image sensor, CCD image sensor, or CMD image sensor). The solid-state imaging element converts light condensed by the lens into an electric signal and outputs the electric signal to the endoscope system 10 as an endoscopic image (electric signal). The endoscope 11 includes a forceps channel. An operator (doctor) can perform various treatments during endoscopy by passing a treatment tool through the forceps channel.

The light source device 15 includes a light source such as a xenon lamp, and supplies observation light (white light, narrow band light, fluorescence, near infrared light, and the like) to the distal end portion of the endoscope 11. The light source device 15 also includes a pump that feeds water or air to the endoscope 11.

The endoscope system 10 controls the light source device 15 and processes an endoscopic image input from the endoscope 11. The endoscope system 10 has functions such as narrow band imaging (NBI), red dichromatic imaging (RDI), texture and color enhancement imaging (TXI), and extended depth of field (EDOF), for example.

In narrow band light imaging, by irradiating violet light with specific wavelength (415 nm) and green light with specific wavelength (540 nm) strongly absorbed by hemoglobin in blood, it is possible to acquire an endoscopic image in which capillaries and microstructures in a mucosal surface layer are emphasized. In red dichroic imaging, by irradiating light of three colors (green, amber, and red) with specific wavelengths, it is possible to acquire an endoscopic image in which the contrast of a deep tissue is enhanced. In texture and color enhancement imaging, an endoscopic image in which three elements, that is, “texture”, “color”, and “brightness” of a mucosal surface under normal light observation are optimized is generated. In extended depth of field, it is possible to acquire an endoscopic image with a wide focus range by combining two images focused on each of a short distance and a long distance.

The endoscope system 10 outputs an endoscopic image obtained by processing an endoscopic image input from the endoscope 11 to the endoscopy support system 30, or outputs an endoscopic image input from the endoscope 11 to the endoscopy support system 30 as it is.

The endoscope position detecting unit 20 is a device for observing the three-dimensional shape of the endoscope 11 inserted into the lumen of the subject. A reception antenna 20a is connected to the endoscope position detecting unit 20. The reception antenna 20a is an antenna for detecting a magnetic field generated by a plurality of magnetic coils built in the endoscope 11.

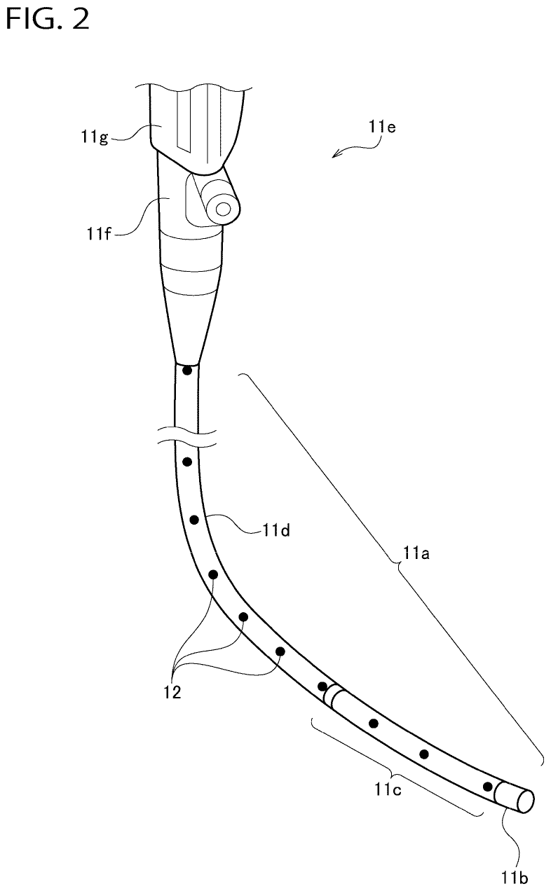

FIG. 2 is a diagram illustrating an example of the endoscope 11 used in the present embodiment. The endoscope 11 includes an elongated tubular insertion portion 11a formed of a flexible member and an operating portion 11e coupled to the proximal end portion of the insertion portion 11a. The insertion portion 11a includes a distal end rigid portion 11b, a curved portion 11c, and a flexible tube portion 11d from the distal end side toward the proximal end side. The proximal end portion of the distal end rigid portion 11b is coupled to the distal end portion of the curved portion 11c, and the proximal end portion of the curved portion 11c is coupled to the proximal end portion of the flexible tube portion 11d.

The operating portion 11e includes a body portion 11f from which the flexible tube portion 11d extends and a grip portion 11g coupled to the proximal end portion of the body portion 11f. The grip portion 11g is gripped by the operator. A universal cord including an imaging electric cable, a light guide, and the like extending from the inside of the insertion portion 11a extends from the operating portion 11e, and is connected to the endoscope system 10 and the light source device 15.

The distal end rigid portion 11b is the distal end portion of the insertion portion 11a and is also the distal end portion of the endoscope 11. The distal end rigid portion 11b incorporates therein a solid-state imaging element, an illumination optical system, an observation optical system, and the like. Illumination light emitted from the light source device 15 is propagated to the distal end face of the distal end rigid portion 11b along the light guide, and is irradiated from the distal end face of the distal end rigid portion 11b toward an observation target in the lumen.

The curved portion 11c is configured by coupling joint rings along the longitudinal axis direction of the insertion portion 11a. The curved portion 11c is curved in a desired direction based on the operation of the operator input to the operating portion 11e, and the position and direction of the distal end rigid portion 11b change depending on the curvature.

The flexible tube portion 11d is a tubular member extending from the body portion 11f of the operating portion 11e, has desired flexibility, and is bent by an external force. The operator inserts the insertion portion 11a into the large intestine of the subject while curving the curved portion 11c or twisting the flexible tube portion 11d.

A plurality of magnetic coils 12 are arranged inside the insertion portion 11a at predetermined intervals (for example, intervals of 10 cm) along the longitudinal direction. Each magnetic coil 12 generates a magnetic field when a current is supplied thereto. The plurality of magnetic coils 12 function as position sensors for detecting each position in the insertion portion 11a.

Returning to FIG. 1, the reception antenna 20a receives magnetic fields transmitted from the plurality of magnetic coils 12 built in the insertion portion 11a of the endoscope 11, and outputs the magnetic fields to the endoscope position detecting unit 20. The endoscope position detecting unit 20 applies the magnetic field intensity of each of the plurality of magnetic coils 12 received by the reception antenna 20a to a predetermined position detection algorithm to estimate the three-dimensional position of each of the plurality of magnetic coils 12. The endoscope position detecting unit 20 generates a three-dimensional endoscope shape of the insertion portion 11a of the endoscope 11 by performing curve interpolation on the estimated three-dimensional positions of the plurality of magnetic coils 12.

A reference plate 20b is attached to the subject (for example, the abdomen of the subject). A body posture sensor for detecting the body posture of the subject is disposed on the reference plate 20b. As the body posture sensor, for example, a three-axis acceleration sensor or a gyro sensor can be used. In FIG. 1, the reference plate 20b is connected to the endoscope position detecting unit 20 by a cable, and the reference plate 20b outputs three-dimensional posture information indicating the posture of the reference plate 20b (that is, the posture of the subject) to the endoscope position detecting unit 20.

A plurality of magnetic coils similar to the plurality of magnetic coils 12 built in the insertion portion 11a of the endoscope 11 may be used as the body posture sensors arranged on the reference plate 20b. In this case, the reception antenna 20a receives magnetic fields transmitted from the plurality of magnetic coils arranged on the reference plate 20b, and outputs the magnetic fields to the endoscope position detecting unit 20. The endoscope position detecting unit 20 applies the magnetic field intensity of each of the plurality of magnetic coils received by the reception antenna 20a to a predetermined posture detection algorithm to generate three-dimensional posture information indicating the posture of the reference plate 20b (that is, the posture of the subject).

The endoscope position detecting unit 20 changes the generated three-dimensional endoscope shape following the change in the three-dimensional posture information. Specifically, the endoscope position detecting unit 20 changes the three-dimensional endoscope shape so as to cancel the change in the three-dimensional posture information. As a result, even in a case where the body posture of the subject is changed during endoscopy, it is possible to always recognize the endoscope shape from a specific viewpoint (for example, a viewpoint at which the abdomen of the subject is viewed vertically from the front side of the abdomen).

The endoscope position detecting unit 20 can acquire an insertion length indicating the length of a portion of the endoscope 11 inserted into the large intestine and an elapsed time (hereinafter, referred to as “insertion time”) since the endoscope 11 has been inserted into the large intestine. For example, the endoscope position detecting unit 20 measures the insertion length with the position at the timing when the operator inputs an examination start operation to the input device 42 as a base point, and measures the insertion time with the timing as a starting point. The endoscope position detecting unit 20 may estimate the position of the anus from the generated three-dimensional endoscope shape and the difference in magnetic field intensity between the magnetic coil inside the body and the magnetic coil outside the body, and may use the estimated position of the anus as the base point of the insertion length.

In order to measure the insertion length with high accuracy, an encoder may be installed near the anus of the subject. The endoscope position detecting unit 20 detects the insertion length with the position of the anus as a base point on the basis of a signal from the encoder.

The endoscope position detecting unit 20 adds the insertion length and the insertion time to the three-dimensional endoscope shape after body posture correction based on the three-dimensional posture information, and outputs the resultant three-dimensional endoscope shape to the endoscopy support system 30.

The endoscopy support system 30 generates endoscopy support information on the basis of the endoscopic image input from the endoscope system 10 and the endoscope shape input from the endoscope position detecting unit 20 and presents the support information to the operator. In addition, the endoscopy support system 30 generates endoscopy history information on the basis of the endoscopic image input from the endoscope system 10 and the endoscope shape input from the endoscope position detecting unit 20 and records the endoscopy history information in the storage device 43.

The display device 41 includes a liquid crystal monitor and an organic EL monitor, and displays an image input from the endoscopy support system 30. The input device 42 includes a mouse, a keyboard, a touch panel, and the like, and outputs operation information input by the operator or the like to the endoscopy support system 30. The storage device 43 includes a storage medium such as an HDD or an SSD, and stores the endoscopy history information generated by the endoscopy support system 30. The storage device 43 may be a dedicated storage device attached to the endoscope system 10, a database in an in-hospital server connected via an in-hospital network, or a database in a cloud server.

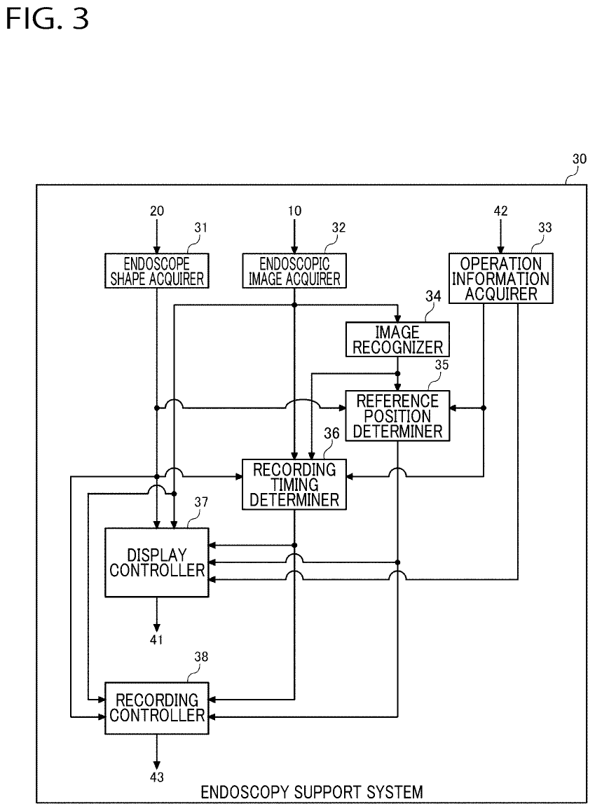

FIG. 3 is a diagram illustrating a configuration example of the endoscopy support system 30 according to the embodiment. The endoscopy support system 30 may be constructed with a processing device dedicated to endoscopy support, or may be constructed with a general-purpose server (which may be a cloud server). Furthermore, the endoscopy support system 30 may be constructed with any combination of a processing device dedicated to endoscopy support, a general-purpose server (which may be a cloud server), and a dedicated image diagnosis device. In addition, the endoscopy support system 30 may be integrally constructed with the endoscope system 10.

The endoscopy support system 30 includes an endoscope shape acquirer 31, an endoscopic image acquirer 32, an operation information acquirer 33, an image recognizer 34, a reference position determiner 35, a recording timing determiner 36, a display controller 37, and a recording controller 38. These components can be implemented by at least one arbitrary processor (for example, CPU and GPU), a memory (for example, DRAM), or other LSIs (for example, FPGA or ASIC) in terms of hardware, and are implemented by a program or the like loaded in a memory in terms of software, but here, functional blocks implemented by cooperation thereof are illustrated. Therefore, it is understood by those skilled in the art that these functional blocks can be implemented in various forms by only hardware, only software, or a combination thereof.

The endoscope shape acquirer 31 acquires an endoscope shape from the endoscope position detecting unit 20. The endoscope shape also includes information of an insertion length and an insertion time. The endoscopic image acquirer 32 acquires an endoscopic image from the endoscope system 10.

The image recognizer 34 includes a plurality of machine learning models for detecting a site of the large intestine, a state in the large intestinal lumen, and a lesion from the endoscopic image. The plurality of machine learning models are generated by machine learning in which a large number of endoscopic images in which annotations are added to various sites, various states, and various lesions are set as a supervised data set. The annotation is added by an annotator with specialized knowledge such as a doctor. For machine learning, CNN, RNN, LSTM, or the like, which is a type of deep learning, can be used.

The large intestine site is roughly classified into rectum, sigmoid colon, descending colon, transverse colon, ascending colon, and cecum in order from the anal side. The image recognizer 34 can input an endoscopic image to a site learning model and detect the site of the large intestine from the endoscopic image. At that time, the image recognizer 34 may specify the site on the basis of detection results of a plurality of endoscopic images continuous in time series. For example, when the same site is detected in a set number or more of frames in endoscopic images of 30 or 60 continuous frames, the image recognizer 34 specifies the site as an official detected site.

Furthermore, the image recognizer 34 may specify the site in consideration of the anteroposterior relationship of the detected site or the endoscope shape acquired from the endoscope position detecting unit 20. For example, the image recognizer 34 specifies whether the moving direction of the endoscope 11 is an insertion direction (anus-cecum) or a removal direction (cecum-anus). In the case of the insertion direction, the image recognizer 34 switches the detected site from the descending colon to the transverse colon in a case where the left colic flexure is detected, and switches the detected site from the transverse colon to the ascending colon in a case where the right colic flexure is detected. In the case of the removal direction, the image recognizer 34 switches the detected site from the ascending colon to the transverse colon in a case where the right colic flexure is detected, and switches the detected site from the transverse colon to the descending colon in a case where the left colic flexure is detected.

Furthermore, the image recognizer 34 may improve the accuracy of site detection in consideration of the three-dimensional position of the distal end rigid portion 11b (hereinafter, referred to as “endoscope distal end portion”) based on the endoscope shape acquired from the endoscope position detecting unit 20. For example, in a case where the position of the endoscope distal end portion estimated from the endoscope shape and the position of the detected site based on the image recognition are inconsistent, the image recognizer 34 discards the detection result based on the image recognition.

Furthermore, the image recognizer 34 can input an endoscopic image to an intraluminal state learning model and determine an intraluminal state from the endoscopic image. The image recognizer 34 can detect, for example, the presence or absence of folds with a predetermined height or more and the presence or absence of diverticula. The image recognizer 34 can also input an endoscopic image to a lesion learning model and detect a lesion candidate from the endoscopic image.

The image recognizer 34 may check the image quality of the endoscopic image prior to image recognition of a detection target. The image recognizer 34 excludes an endoscopic image determined to have poor image quality (for example, blurring, out-of-focus, and luminance abnormality (for example, halation)) from targets of the image recognition of the detection target.

The reference position determiner 35 determines an endoscope shape to be set as a reference position from endoscope shapes continuously acquired from the endoscope position detecting unit 20. The reference position is determined to be a position at the time of reaching the deepest part in colonoscopy. The deepest part in colonoscopy is usually the cecum. Depending on the operator, the endoscope 11 may be inserted into the ileum. In addition, depending on the subject, the endoscope 11 cannot be inserted into the cecum, and the ascending colon may be the deepest part in colonoscopy.

For example, the reference position determiner 35 determines a position at which the insertion length acquired from the endoscope position detecting unit 20 is the longest as the position at the time of reaching the deepest part. In addition, the reference position determiner 35 may determine the capturing position of the endoscopic image in which the cecum is detected by the image recognizer 34 as the position at the time of reaching the deepest part. Furthermore, the reference position determiner 35 may determine the position at the timing when the operator inputs an insertion completion operation to the input device 42 as the position at the time of reaching the deepest part.

The recording timing determiner 36 determines the recording timing of the endoscopic image and the endoscope shape. For example, the recording timing determiner 36 determines the timing when the operator presses a capture button (release button) of the operating portion 11e as the recording timing. In a case where a microphone is installed on the pharynx or the like of the operator, the operator can also give an instruction about the recording timing by voice. Furthermore, the recording timing determiner 36 may determine the capturing timing of the endoscopic image in which the lesion candidate is detected by the image recognizer 34 as the recording timing.

In addition, the recording timing determiner 36 may automatically determine the recording timing on the basis of a predetermined rule. Automatic records of the endoscopic image and the endoscope shape are utilized to generate an examination digest. In general, in colonoscopy, observation and treatment are performed while the endoscope 11 is inserted into the cecum and then removed toward the anus. For example, the recording timing determiner 36 may set the recording timing every time the insertion length increases by a predetermined interval (for example, several cm). Furthermore, the recording timing determiner 36 may set the recording timing every time a predetermined removal time elapses.

The recording timing determiner 36 may change the frequency of automatic recording depending on the site or the intraluminal state detected by the image recognizer 34. For example, the recording timing determiner 36 increases the frequency of automatic recording while the endoscope passes through a site where a lesion is likely to occur. In addition, the recording timing determiner 36 increases the frequency of automatic recording while the endoscope passes through a part where the intraluminal state is inferior. The site in which the frequency of automatic recording is increased may be set in advance on the basis of the medical history of the subject and epidemiological knowledge.

The recording timing determiner 36 may determine at least one or all of the timing based on the operation of the operator, the timing based on the detection of the lesion candidate by the image recognizer 34, and the timing based on automatic setting as the recording timing.

When displaying the examination information including the endoscopic image, the display controller 37 can simultaneously display two endoscope shapes, that is, the endoscope shape at the time of reaching the deepest part and the endoscope shape at a specific recording timing on the display device 41. The display controller 37 may display the two endoscope shapes when a predetermined operation is input to the input device 42. The display controller 37 may display the two endoscope shapes in one graph or may display the endoscope shapes side by side in two graphs.

The display controller 37 can display the two endoscope shapes in real time during examination. In a case where a lesion candidate is detected by the image recognizer 34 during examination, the display controller 37 superimposes a mark surrounding the lesion candidate on the endoscopic image in which the lesion candidate is detected. As a result, the risk of overlooking the lesion or the like can be reduced. Alert voice may be output from a speaker.

Meanwhile, during endoscopy, the subject may change the body posture in accordance with instructions from the operator or slightly move by himself or herself. The body posture is changed to facilitate insertion and observation of the endoscope 11. For this reason, the position and direction of the endoscope shape may deviate from the coordinate space based on the position and direction of an examination room or an examination table (bed).

In order to handle this, in the example illustrated in FIG. 1, the reference plate 20b is attached to the subject, the position and direction of the subject are detected, and the position and direction of the endoscope shape are corrected on the basis of the detection result. In this respect, in many cases, colonoscopy is performed without using the reference plate 20b. Even in that case, if the insertion length is equal to or more than a predetermined value, the position and direction of the endoscope shape can be corrected by matching the arrangement model of the large intestinal lumen and the endoscope shape. In particular, at the time of removal, it is effective since the shape of the insertion portion 11a substantially matches the arrangement of the large intestinal lumen. This correction may be performed by the endoscope position detecting unit 20 or may be performed by the endoscopy support system 30.

The recording controller 38 records the examination information including the endoscopic image acquired during examination in the storage device 43 in association with the endoscope shape at the time of reaching the deepest part and the endoscope shape at at least one recording timing. Since the shape of the endoscope shape at the time of reaching the deepest part is common in one case, it is only required that recording associated with the case can be performed. The format of the data of the endoscope shape to be recorded is not limited. For example, it may be a mathematical expression for calculating a shape, point cloud data for indicating a shape, or image data viewed from one or a plurality of directions.

In a case where the doctor confirms the examination information after endoscopy, the examination information recorded in the storage device 43 is read into the endoscopy support system 30. The display controller 37 displays, on the display device 41, two types of endoscope shapes, that is, the endoscope shape at the time of reaching the deepest part associated with the examination information and the endoscope shape at at least one recording timing. The examination information may be appropriately selected, reformatted, or transferred to another database, and then the examination information may be displayed on a monitor of another PC. Also in this case, two types of endoscope shapes, that is, the endoscope shape at the time of reaching the deepest part and the endoscope shape at at least one recording timing are simultaneously displayed on the monitor.

As described above, in endoscopy, Each of the display controller 37 and the recording controller 38 can acquire a first endoscope shape when the endoscope distal end portion is located at a predetermined site, an endoscopic image captured in the endoscopy, and a second endoscope shape at the time of capturing the endoscopic image. As a result, it is possible to present the endoscopic image simultaneously with the second endoscope shape at the time of capturing the endoscopic image and the first endoscope shape when the endoscope distal end portion is located at the predetermined site. The first endoscope shape and the second endoscope shape are different from each other. The predetermined site may be the deepest part at the time of endoscopy. In this case, in the endoscopy, the endoscope shape when the endoscope distal end portion is located at the deepest part can be determined as the first endoscope shape. The predetermined site may be the cecum. In this case, the first endoscope shape in a case where the deepest part at the time of endoscopy is the cecum can be determined.

In colonoscopy, at the time of insertion, the insertion portion 11a of the endoscope 11 is inserted into the tortuous intestinal tract of the large intestine. At this time, the internal arrangement of the unfixed intestinal tract part changes or expands and contracts, and thus, even for the same patient, the insertion shape of the endoscope 11 may change depending on a doctor or an insertion skill, or for each examination. However, the internal arrangement of the part fixed to the patient's body like the ascending colon and the descending colon does not largely change. The cecum part, which is the end of the large intestine, is at the end of such a fixed part, and the internal position hardly changes.

The cecum is the deepest part of the large intestine, and is also a target to be reached by the doctor in colonoscopy. In practice, the endoscope 11 may be inserted into the ileum, which is the end of the small intestine, but the cecum is one of sites that can be a relatively stable reference point at the time of examination also from the point of being at the end of the ascending colon, which is the fixed part. In addition, in an inspection case where the endoscope cannot be inserted into the cecum because of insertion difficulty or the like, the deepest part is one of sites that can be a reference point.

Since the insertion shape of the insertion portion 11a of the endoscope 11 to the cecum or the deepest part can be known, it is possible to confirm the arrangement of the large intestine tract of the patient and the degree of insertion of the endoscope 11 in the examination, which have individual differences. Therefore, it is effective to determine the endoscope shape when the endoscope distal end portion is located at the cecum or the deepest part as the first endoscope shape.

On the other hand, when the endoscope 11 is removed from the deepest part, for example, the cecum, even if the endoscope is inserted in the bent intestinal tract, the bent part is extended and the degree of bending is reduced. As a result, the intestinal tract at the time of removal has a shape with less bending based on the original arrangement. It is considered that the shape arrangement of the intestinal tract is closer to a more stable shape arrangement than that at the time of insertion. At that time, it is considered that the distal end of the insertion portion 11a of the endoscope 11 passes through a route or draws a trajectory with less variation than at the time of insertion. In addition, a route through which the distal end of the insertion portion 11a of the endoscope 11 passes or a trajectory drawn by the distal end is considered to be a route or a trajectory substantially determined by the shape of the insertion portion 11a of the endoscope 11 at the time of reaching the cecum even if the route or the trajectory is not determined to be one stable route or trajectory.

The reference position determiner 35 determines that the endoscope distal end portion is located at a predetermined site on the basis of at least one of the endoscopic image, the endoscope shape, or the insertion length of the endoscope. Each of the display controller 37 and the recording controller 38 acquires an endoscope shape when the endoscope distal end is located at a predetermined part as the first endoscope shape. In this case, the first endoscope shape can be automatically acquired. Each of the display controller 37 and the recording controller 38 may acquire, as the first endoscope shape, the endoscope shape at the timing when the reference position determiner 35 acquires an endoscope insertion completion signal based on the operation of the operator. In this case, the first endoscope shape that meets the intention of the operator can be acquired.

The recording controller 38 can record the first endoscope shape, the captured endoscopic image, and the second endoscope shape corresponding to the endoscopic image in the storage device 43 in association with each other. With this configuration, after the examination, the endoscopic image can be presented simultaneously with the second endoscope shape at the time of capturing the endoscopic image and the first endoscope shape when the endoscope distal end portion is located at the predetermined site.

The display controller 37 can simultaneously display the first endoscope shape and the second endoscope shape on the display device 41. With this configuration, the operator and the doctor can intuitively grasp the relative position of the second endoscope shape at the time of capturing the endoscopic image. At that time, the display controller 37 can display the first endoscope shape and the second endoscope shape on one graph. With this configuration, the operator and the doctor can more accurately grasp the relative position of the second endoscope shape at the time of capturing the endoscopic image. In addition, the display controller 37 can simultaneously display the first endoscope shape, the second endoscope shape, and the endoscopic image corresponding to the second endoscope shape on the display device 41. With this configuration, the operator and the doctor can simultaneously grasp the endoscopic image and the relative position of the second endoscope shape at the time of capturing the endoscopic image.

As a result, at the time of removal, the arrangement of the distal end of the first endoscope shape and the distal end of the second endoscope shape on the intestinal tract is displayed with relatively good reproducibility. Therefore, on the basis of the first endoscope shape in which the endoscope is disposed in a deeper part and the distal end position thereof, the position and arrangement in the intestinal tract of the distal end position of the second endoscope shape can be confirmed with relatively good reproducibility from the second endoscope shape at a position where the endoscope has been removed from the distal end position of the first endoscope shape. These results can be confirmed during and after the examination. Therefore, it can be position specifying information at the time of recording the position of the lesion or the like during the examination, and can be guide information with good reproducibility at the time of approaching the same lesion or the like at the time of the subsequent examination or treatment. In particular, by using an image captured when the endoscope has the second endoscope shape, it is easier to specify the position of the lesion or the like and to re-approach the lesion or the like, and it is possible to reliably determine the position.

Furthermore, in a case where the second endoscope shape is acquired at the time of insertion of the endoscope 11, recording the second endoscope shape as a record at the time of insertion into an insertion difficult site or the like in comparison with the deepest part or the shape at the time of reaching the cecum is useful as a record of an individual examination or patient in that it can be confirmed together with the distal end position or the insertion shape and the event at the position.

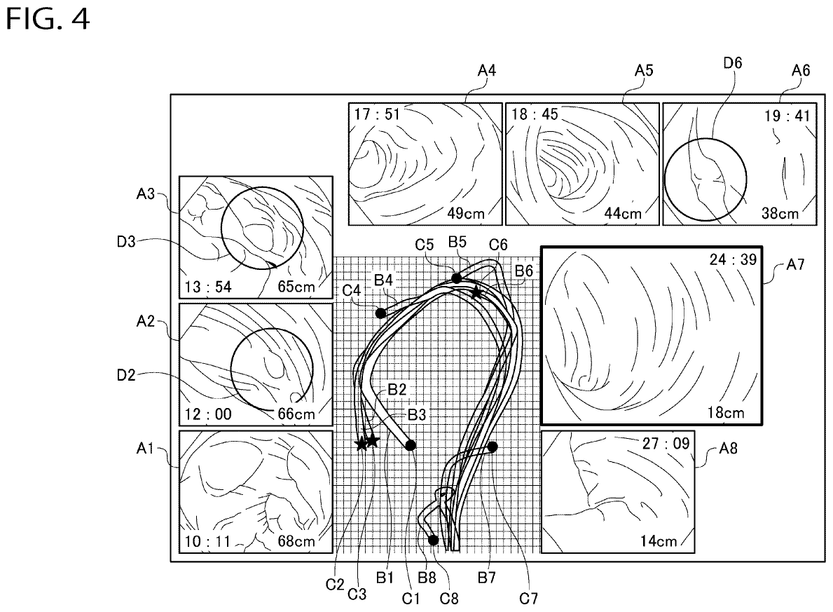

FIG. 4 is a diagram illustrating a first screen example displayed on the display device 41. The following screen examples assume a screen example when the doctor confirms examination information after examination, but similar screen display can be made during the examination. In the case of during the examination, every time the operator captures an endoscopic image, the endoscopic image and the endoscope shape at the time of capturing are added to the screen.

In the first screen example, an endoscope shape B1 at the time of reaching the cecum and a plurality of endoscope shapes B2 to B8 at the time of capturing are simultaneously displayed on a graph disposed at the center. The display controller 37 aligns and displays specific portions of the endoscope shape B1 and specific portions of the plurality of endoscope shapes B2-B8. The specific portion may be a site (the anus in colonoscopy) corresponding to the insertion port of the subject into which the endoscope 11 is inserted. With this configuration, with respect to the endoscope shape B1 (first endoscope shape) at the time of reaching the cecum, the plurality of endoscope shapes B2 to B8 (second endoscope shapes) at the time of capturing can be arranged at positions based on the actual state. Marks C1 to C8 indicating capturing positions are added to distal end portions of the endoscope shape B1 at the time of reaching the cecum and the plurality of endoscope shapes B2 to B8 at the time of capturing.

A plurality of endoscopic images A1 to A8 are displayed so as to surround the graph disposed at the center. The lower left endoscopic image A1 is an endoscopic image at the time of reaching the cecum, and the plurality of endoscopic images A2 to A8 are arranged clockwise in order of a removal direction. In each of the endoscopic images A1 to A8, the insertion length and the insertion time (elapsed time from the start of insertion) are displayed.

The display controller 37 acquires the plurality of endoscopic images A2 to A8 and the plurality of endoscope shapes B2 to B8 corresponding to the plurality of endoscopic images A2 to A8, respectively, and displays the plurality of endoscopic images A2 to A8 and the plurality of endoscope shapes B2 to B8 on the display device 41 simultaneously with the endoscope shape B1. By displaying the plurality of endoscopic images A2 to A8, the plurality of endoscope shapes B2 to B8 (second endoscope shapes), and the endoscope shape B1 (first endoscope shape) as a list, the operator and the doctor can easily grasp the outline of endoscopy.

In a case where the correction of the body posture change of the subject is not performed in the endoscope position detecting unit 20, the display controller 37 estimates the change in the position and direction of the subject from the change in the acquired endoscope shape, and positionally and directionally aligns the endoscope shape B1 with the plurality of endoscope shapes B2 to B8 on the basis of the estimation result. The display controller 37 simultaneously displays the endoscope shape B1 and the plurality of endoscope shapes B2 to B8 after positional and directional alignment on the display device 41. With this configuration, it is possible to continuously display the endoscope shape B1 (first endoscope shape) at the time of reaching the cecum and the plurality of endoscope shapes B2 to B8 (second endoscope shapes) at the time of capturing as the endoscope shapes from a specific viewpoint (for example, viewpoint at which the abdomen of the subject is viewed vertically from the front side of the abdomen) while arranging the endoscope shapes B1 (first endoscope shape) and the plurality of endoscope shapes B2 to B8 (second endoscope shapes) at positions based on the actual state.

In the example illustrated in FIG. 4, lesion candidates are detected in three endoscopic images A2, A3, and A6. Marks D2, D3, and D6 each of which circles each lesion candidate are superimposed on the three endoscopic images A2, A3, and A6, respectively. Among the marks C1 to C8 indicating the capturing positions of the plurality of endoscope shapes B1 to B8, the marks C1, C4 to C5, and C7 to C8 indicating the capturing positions of the endoscope shapes B1, B4 to B5, and B7 to B8 associated with the endoscopic images A1, A4 to A5, and A7 to A8 in which no lesion candidate is detected are displayed as circle marks, and the marks C2, C3, and C6 indicating the capturing positions of the endoscope shapes B2, B3, and B6 associated with the endoscopic images A2, A3, and A6 in which the lesion candidate is detected are displayed as star marks.

The display controller 37 acquires the endoscopic images A2, A3, and A6 in which the lesion candidate is detected and the endoscope shapes B2, B3, and B6 at the time of capturing the endoscopic images A2, A3, and A6 in which the lesion candidate is detected. With this configuration, it is possible to acquire the endoscopic image with high importance and the endoscope shape as the display target. The display controller 37 simultaneously displays the endoscope shapes B2, B3, and B6 and the marks C2, C3, and C6 arranged at the distal end portions of the endoscope shapes B2, B3, and B6 and indicating that the lesion candidate is detected on the display device 41. With this configuration, the operator and the doctor can more easily grasp the capturing position of the endoscopic image in which the lesion candidate is detected.

In the example illustrated in FIG. 4, an example is illustrated in which the endoscope shape in a state where the subject is in a supine posture on the examination table is viewed from the ceiling viewpoint. As will be described later, an endoscope shape viewed from viewpoints from two to three directions may be displayed, or an endoscope shape viewed from an oblique direction may be displayed in a perspective view. By displaying the endoscope shape at the time of removal in this manner, it is possible to clearly present the site in the large intestinal lumen where the endoscope distal end portion is located.

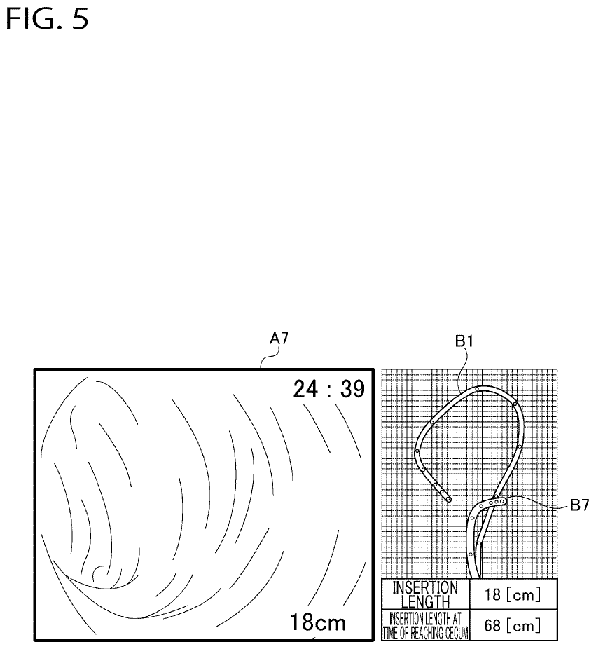

FIG. 5 is a diagram illustrating a second screen example displayed on the display device 41. The second screen example is a screen example to which the screen is transitioned in a case where the endoscopic image A7 is selected by a click operation or a touch operation of a user such as a doctor in the first screen example illustrated in FIG. 4. The endoscopic image A7 attracting the attention of the user is displayed on the left side of the screen, and one graph in which the endoscope shape B7 at the time of capturing the endoscopic image A7 and the endoscope shape B1 at the time of reaching the cecum are simultaneously plotted is displayed on the right side of the screen. The insertion length (18 [cm]) at the time of capturing the endoscopic image A7 and the insertion length (68 [cm]) at the time of reaching the cecum are displayed at the bottom of the graph. The graph on the right side and the table of the insertion length may be displayed only when a predetermined operation is performed on the endoscopic image A7 by the user in order to simplify the screen display.

The display controller 37 displays the endoscopic image A7 selected by the user from the plurality of endoscopic images A2 to A8 and the endoscope shape B7 corresponding to the selected endoscopic image A7 on the display device 41 simultaneously with the endoscope shape B1. With this configuration, it is possible to generate a screen focused on information of the endoscopic image attracting the attention of the user. At that time, the display controller 37 may display the insertion length corresponding to the endoscope shape B1 and the insertion length corresponding to the endoscope shape B7 on the display device 41 simultaneously with the endoscope shape B1 and the endoscope shape B7. By displaying the insertion length simultaneously, the amount of information to be presented to the user can be increased. The display controller 37 may display the insertion time corresponding to the endoscope shape B1 and the insertion time corresponding to the endoscope shape B7 on the display device 41 simultaneously with the endoscope shape B1 and the endoscope shape B7. By displaying the insertion time simultaneously, the amount of information to be presented to the user can be increased.

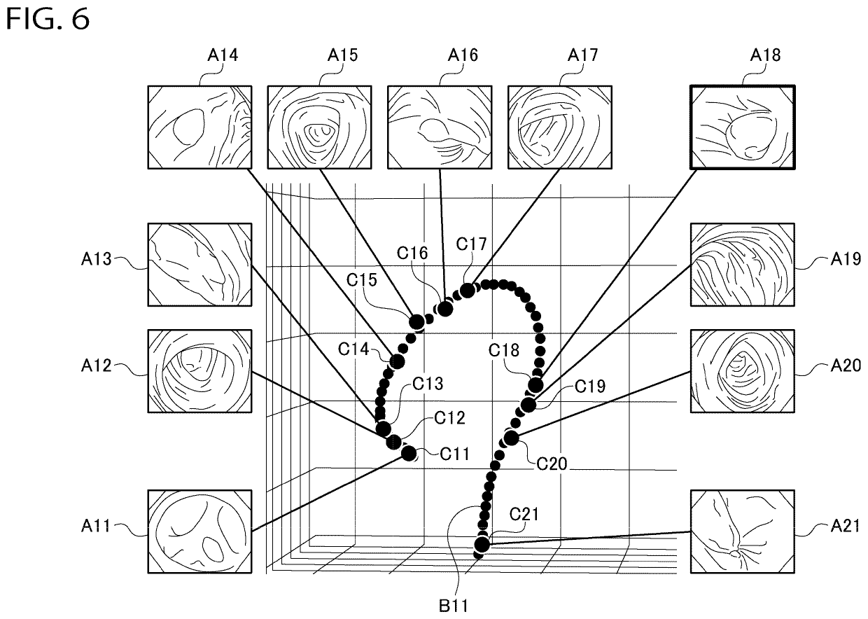

FIG. 6 is a diagram illustrating a third screen example displayed on the display device 41. In the third screen example, only an endoscope shape B11 at the time of reaching the cecum is displayed on a three-dimensional graph disposed at the center. A plurality of endoscopic images A11 to A21 are displayed so as to surround the graph disposed at the center. The plurality of endoscopic images A11 to A21 may be thumbnail images. The lower left endoscopic image A11 is an endoscopic image at the time of reaching the cecum, and the plurality of endoscopic images A12 to A21 are arranged clockwise in order of the removal direction. Marks C11 to C21 indicating capturing positions of the plurality of endoscopic images A11 to A21 are added on the endoscope shape B11 at the time of reaching the cecum.

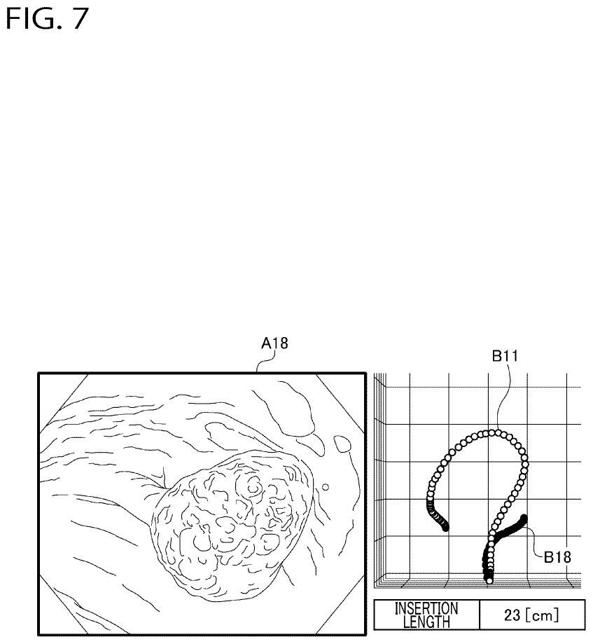

FIG. 7 is a diagram illustrating a fourth screen example displayed on the display device 41. The fourth screen example is a screen example to which the screen is transitioned in a case where the endoscopic image A18 is selected by the user in the third screen example illustrated in FIG. 6. The endoscopic image A18 attracting the attention of the user is displayed on the left side of the screen, and one graph in which an endoscope shape B18 at the time of capturing the endoscopic image A18 and the endoscope shape B11 at the time of reaching the cecum are simultaneously plotted is displayed on the right side of the screen. The insertion length (23 [cm]) at the time of capturing the endoscopic image A18 is displayed at the bottom of the graph. The screen example illustrated in FIG. 6 and the screen example illustrated in FIG. 7 may be displayed in one screen.

The display controller 37 can switch between a first display mode in which the endoscope shape B11, the plurality of endoscopic images A11 to A21, and the marks C11 to C21 arranged on the endoscope shape B11 and indicating capturing positions of the plurality of endoscopic images A11 to A21 are displayed on the display device 41, and a second display mode in which the endoscopic image A18 selected by the user, the endoscope shape B18 corresponding to the selected endoscopic image A18, and the endoscope shape B11 are simultaneously displayed on the display device 41. With this configuration, the visibility or operability of the user can be improved.

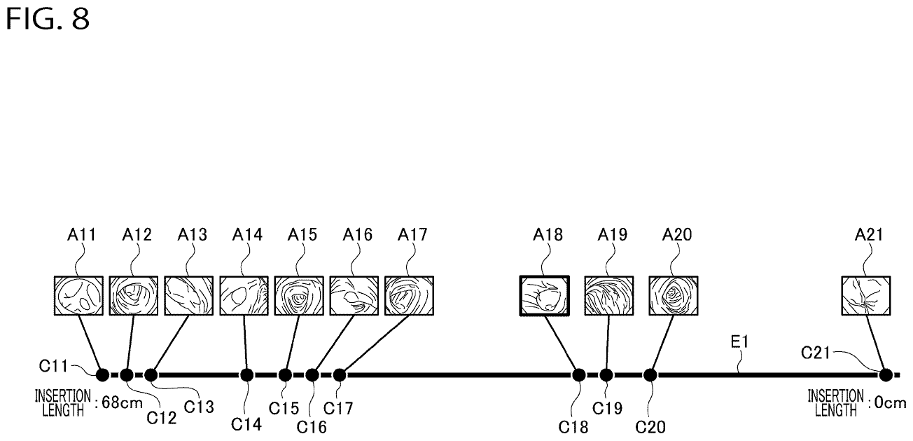

FIG. 8 is a diagram illustrating a fifth screen example displayed on the display device 41. In the fifth screen example, an insertion length line E1 generated by straightening the endoscope shape B11 at the time of reaching the cecum illustrated in FIG. 6 is displayed, and the plurality of endoscopic images A11 to A21 are displayed above the insertion length line E1 in parallel with the insertion length line E1. The plurality of endoscopic images A11 to A21 may be thumbnail images. The leftmost endoscopic image A11 is an endoscopic image at the time of reaching the cecum, and the plurality of endoscopic images A12 to A21 are arranged in order of the removal direction toward the right. The marks C11 to C21 indicating capturing positions of the plurality of endoscopic images A11 to A21 are added on the insertion length line E1. When any one of the endoscopic images A11 to A21 is selected by the user, the screen transitions to the screen example illustrated in FIG. 7.

The display controller 37 can switch between a third display mode in which the endoscope shape B11, the plurality of endoscopic images A11 to A21, and the marks C11 to C21 arranged on the insertion length line E1 generated by straightening the endoscope shape B11 and indicating capturing positions of the plurality of endoscopic images A11 to A21 are displayed on the display device 41, and the second display mode. With this configuration, the visibility or operability of the user can be improved.

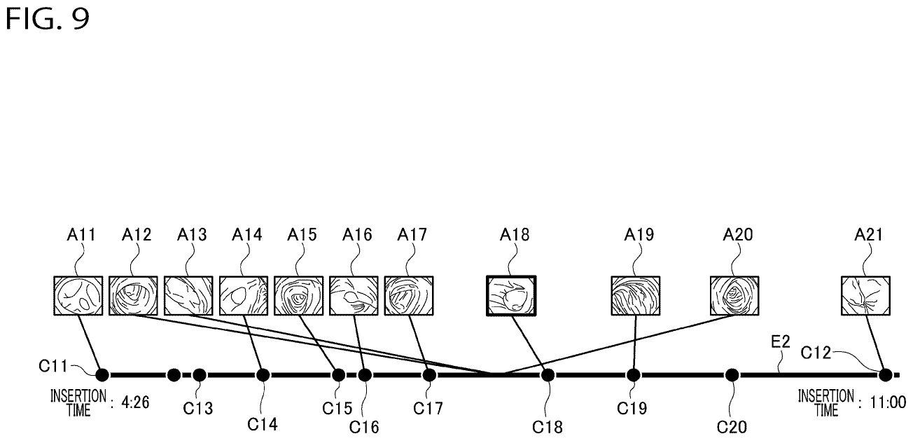

FIG. 9 is a diagram illustrating a sixth screen example displayed on the display device 41. In the sixth screen example, an insertion time line E2 generated by straightening the endoscope shape B11 at the time of reaching the cecum illustrated in FIG. 6 is displayed, and the plurality of endoscopic images A11 to A21 are displayed above the insertion time line E2 in parallel with the insertion time line E2. The plurality of endoscopic images A11 to A21 may be thumbnail images. The leftmost endoscopic image A11 is an endoscopic image at the time of reaching the cecum, and the plurality of endoscopic images A12 to A21 are arranged in order of the removal direction toward the right. The marks C11 to C21 indicating capturing timings of the plurality of endoscopic images A11 to A21 are added on the insertion time line E2. In the example illustrated in FIG. 9, the elapsed time from the start of insertion to the insertion time at the time of reaching the cecum is 4:26, and the insertion time is counted up to the completion of removal. When any one of the endoscopic images A11 to A21 is selected by the user, the screen transitions to the screen example illustrated in FIG. 7.

The display controller 37 can switch between a fourth display mode in which the endoscope shape B11, the plurality of endoscopic images A11 to A21, and the marks C11 to C21 arranged on the insertion time line E2 generated by straightening the endoscope shape B11 and indicating capturing timings of the plurality of endoscopic images A11 to A21 are displayed on the display device 41, and the second display mode. With this configuration, the visibility or operability of the user can be improved.

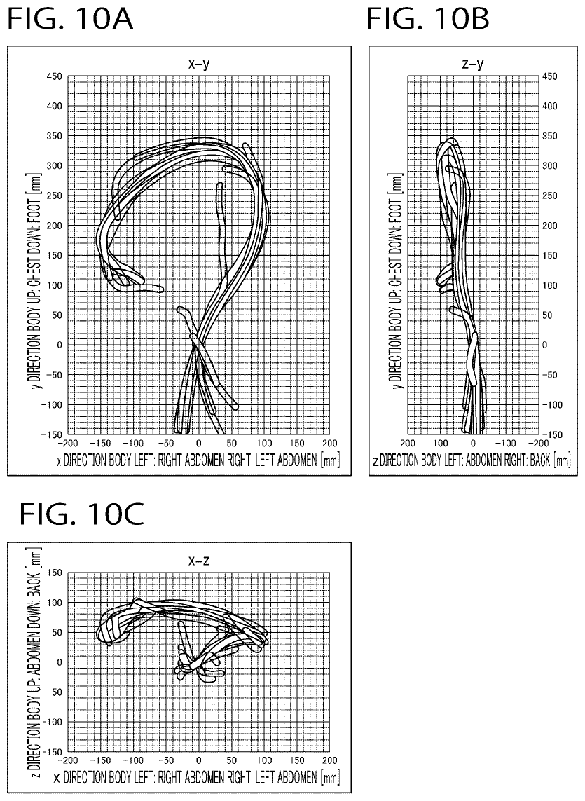

FIGS. 10A to 10C are diagrams illustrating examples in which a plurality of endoscope shapes are displayed as trigonometric projections in three directions. In FIGS. 10A to 10C, in the subject in the supine posture, the y direction is defined as a longitudinal direction, the x direction is defined as a lateral direction, and the z direction is defined as a thickness direction. FIG. 10A illustrates an example in which a plurality of endoscope shapes are plotted on the x-y coordinate. The left side in the x-axis is a right abdominal direction, the right side in the x-axis is a left abdominal direction, the upper side in the y-axis is a chest direction, and the lower side in the y-axis is a foot direction. FIG. 10B illustrates an example in which a plurality of endoscope shapes are plotted on the z-y coordinate. The left side in the z-axis is an abdominal direction, the right side in the z-axis is a back direction, the upper side in the y-axis is the chest direction, and the lower side in the y-axis is the foot direction. FIG. 10C illustrates an example in which a plurality of endoscope shapes are plotted on the x-z coordinate. The left side in the x-axis is the right abdominal direction, the right side in the x-axis is the left abdominal direction, the upper side in the z-axis is the abdominal direction, and the lower side in the z-axis is the back direction.

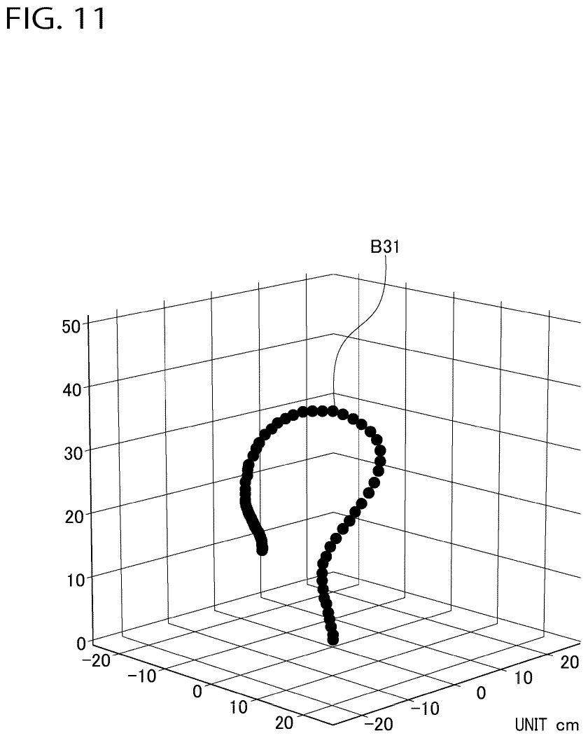

FIG. 11 is a diagram illustrating an example in which a three-dimensional endoscope shape B31 at the time of reaching the cecum is displayed in a bird's eye perspective view. In FIG. 11, display is made using a coordinate system with the position corresponding to the anus, which is an insertion start point into the body, as the origin. In addition, the coordinate system has actual dimensional scales. It is assumed that the viewpoint direction and scale can be appropriately changed and set to the direction and size desired by the operator to confirm the three-dimensional endoscope shape B31. A plurality of endoscope shapes at the time of capturing illustrated in FIG. 4 may also be simultaneously displayed on the same three-dimensional graph. In addition, the coordinate system may be set in any manner, and the dimensions may also be displayed in any manner. For example, the coordinate system does not need to be displayed, the origin does not need to be the anal position, the dimensions do not need to be displayed, or only the scale may be displayed.

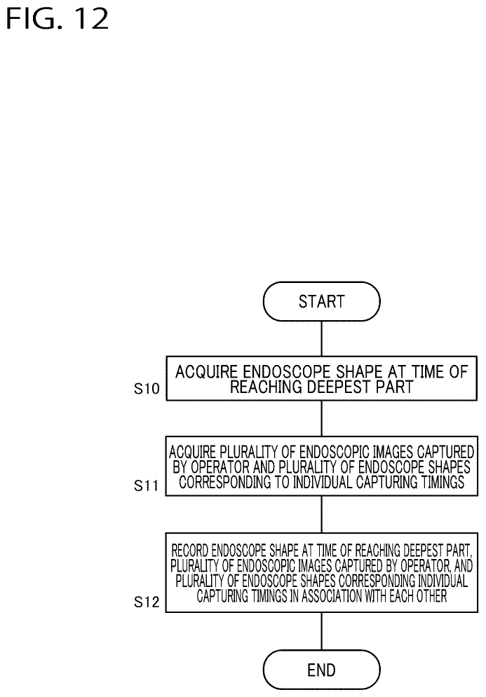

FIG. 12 is a flowchart illustrating an operation example of the endoscopy support system 30 according to the embodiment at the end of an examination. The recording controller 38 acquires an endoscope shape at the time of reaching the deepest part (S10). The recording controller 38 acquires a plurality of endoscopic images captured by the operator and a plurality of endoscope shapes corresponding to the individual capturing timings (S11). The recording controller 38 records the endoscope shape at the time of reaching the deepest part, the plurality of endoscopic images captured by the operator, and the plurality of endoscope shapes corresponding the individual capturing timings in the storage device 43 in association with each other (S12).

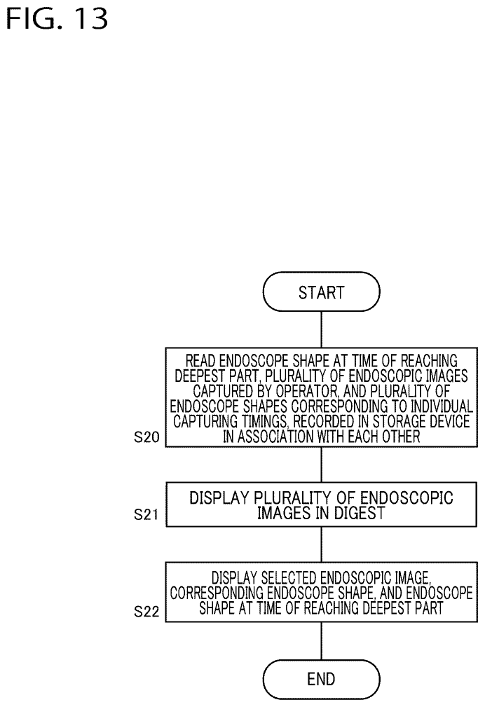

FIG. 13 is a flowchart illustrating an operation example of the endoscopy support system 30 according to the embodiment at the time of confirming examination information. The display controller 37 reads, from the storage device 43, the endoscope shape at the time of reaching the deepest part, the plurality of endoscopic images captured by the operator, and the plurality of endoscope shapes corresponding to the individual capturing timings, which are recorded in the storage device 43 in association with each other (S20). The display controller 37 displays the plurality of read endoscopic images on the display device 41 in a digest (S21). The display controller 37 displays an endoscopic image selected by the user, the endoscope shape corresponding to the endoscopic image, and the endoscope shape at the time of reaching the deepest part on the display device 41 (S22).

As described above, according to the present embodiment, by simultaneously displaying the endoscope shape at the time of reaching the deepest part and the endoscope shape at the capturing timing, it is easy for the endoscope 11 to reaccess the lesion or the lesion candidate. The operator can more accurately grasp the position in the large intestinal lumen where the lesion or lesion candidate is present, and reaccess is facilitated. In addition, the visibility or operability of the user can be improved by adopting various display modes described above.

The present disclosure has been described above on the basis of a plurality of embodiments. It is to be understood by those skilled in the art that these embodiments are merely examples, that various modifications can be made to combinations of the individual components and the individual processing processes, and that such modifications are also within the scope of the present disclosure.

In the above embodiment, an example has been described in which the endoscope shape is estimated by incorporating a plurality of magnetic coils in the endoscope 11. In this regard, the endoscope shape may be estimated by incorporating a plurality of shape sensors in the endoscope 11. The shape sensor may be, for example, a fiber sensor that detects a bent shape from the curvature of a specific location using an optical fiber. The fiber sensor includes, for example, an optical fiber disposed along the longitudinal direction of the insertion portion 11a, and the optical fiber includes a plurality of optical detectors along the longitudinal direction. The endoscope shape is estimated on the basis of a change in the amount of light detected by each optical detector when detection light is supplied from a detection light emitting device to the optical fiber and the detection light is propagating through the optical fiber.

What is claimed is:

1. An endoscopy support system comprising:
one or more processors comprising hardware, wherein the one or more processors are configured to:
acquire, in endoscopy, a first curved shape of an endoscope insertion portion when an endoscope distal end portion is located at a predetermined site, an endoscopic image captured in the endoscopy, and a second curved shape of an endoscope insertion portion when the endoscopic image is captured, and
display the first curved shape of the endoscope insertion portion and the second curved shape of the endoscope insertion portion simultaneously on a monitor.

2. The endoscopy support system according to claim 1, wherein
the one or more processors are configured to display the first curved shape of the endoscope insertion portion, the second curved shape of the endoscope insertion portion, and the endoscopic image simultaneously on the monitor.

3. The endoscopy support system according to claim 1, wherein
the one or more processors are configured to align and display a specific portion of the first curved shape of the endoscope insertion portion and a specific portion of the second curved shape of the endoscope insertion portion.

4. The endoscopy support system according to claim 1, wherein
the one or more processors are configured to align and display a site corresponding to an insertion port of a subject in the first curved shape of the endoscope insertion portion and a site corresponding to the insertion port in the second curved shape of the endoscope insertion portion.

5. The endoscopy support system according to claim 1, wherein
the one or more processors are configured are configured to:
estimate a change in position and direction of a subject, and
positionally and directionally align the first curved shape of the endoscope insertion portion and the second curved shape of the endoscope insertion portion based on an estimation result, and simultaneously display the first curved shape of the endoscope insertion portion and the second curved shape of the endoscope insertion portion on the monitor.

6. The endoscopy support system according to claim 1, wherein
the one or more processors are configured to acquire the endoscopic image in which a lesion candidate is detected and the second curved shape of the endoscope insertion portion when the endoscopic image in which the lesion candidate is detected is captured.

7. The endoscopy support system according to claim 1, wherein
the predetermined site is a deepest part at the time of endoscopy.

8. The endoscopy support system according to claim 1, wherein
the predetermined site is a cecum.

9. The endoscopy support system according to claim 1, wherein
the one or more processors are configured to:
determine that the endoscope distal end portion is located at the predetermined site based on at least one of an endoscopic image, a curved shape of an endoscope insertion portion, or an insertion length of an endoscope, and
acquire a curved shape of the endoscope insertion portion when the endoscope distal end portion is located at the predetermined site as the first curved shape of the endoscope insertion portion.

10. The endoscopy support system according to claim 1, wherein
the one or more processors are configured to acquire a curved shape of an endoscope insertion portion at a timing when an endoscope insertion completion signal based on an operation of an operator is acquired as the first curved shape of the endoscope insertion portion.

11. The endoscopy support system according to claim 1, wherein
the first curved shape of the endoscope insertion portion and the second curved shape of the endoscope insertion portion are different from each other.

12. The endoscopy support system according to claim 2, wherein
the one or more processors are configured to display an insertion length corresponding to the first curved shape of the endoscope insertion portion and an insertion length corresponding to the second curved shape of the endoscope insertion portion on the monitor simultaneously with the first curved shape of the endoscope insertion portion and the second curved shape of the endoscope insertion portion.

13. The endoscopy support system according to claim 2, wherein
the one or more processors are configured to display an elapsed time from start of insertion corresponding to the first curved shape of the endoscope insertion portion and an elapsed time from the start of insertion corresponding to the second curved shape of the endoscope insertion portion on the monitor simultaneously with the first curved shape of the endoscope insertion portion and the second curved shape of the endoscope insertion portion.

14. The endoscopy support system according to claim 2, wherein
the one or more processors are configured to:
acquire a plurality of the endoscopic images and a plurality of the second curved shapes of the endoscope insertion portion corresponding to each of a plurality of the endoscopic images, and
display a plurality of the endoscopic images and a plurality of the second curved shapes of the endoscope insertion portion on the monitor simultaneously with the first curved shape of the endoscope insertion portion.

15. The endoscopy support system according to claim 2, wherein
the one or more processors are configured to:
acquire a plurality of the endoscopic images and a plurality of the second curved shapes of the endoscope insertion portion corresponding to each of a plurality of the endoscopic images, and
display the endoscopic image selected from a plurality of the endoscopic images by a user and the second curved shape of the endoscope insertion portion corresponding to the endoscopic image selected on the monitor simultaneously with the first curved shape of the endoscope insertion portion.

16. The endoscopy support system according to claim 2, wherein
the one or more processors are configured to be switchable between
a first display mode in which the first curved shape of the endoscope insertion portion, and a plurality of the endoscopic images, and marks arranged on the first curved shape of the endoscope insertion portion and indicating capturing positions of a plurality of the endoscopic images are displayed on the monitor and
a second display mode in which an endoscopic image selected by a user, a second curved shape of an endoscope insertion portion corresponding to the endoscopic image selected, and the first curved shape of the endoscope insertion portion are simultaneously displayed on the monitor.

17. The endoscopy support system according to claim 2, wherein
the one or more processors are configured to be switchable between
a third display mode in which the first curved shape of the endoscope insertion portion, a plurality of the endoscopic images, and marks arranged on an insertion length line obtained by straightening the first curved shape of the endoscope insertion portion and indicating capturing positions of a plurality of the endoscopic images are displayed on the monitor and
a second display mode in which an endoscopic image selected by a user, a second curved shape of an endoscope insertion portion corresponding to the endoscopic image selected, and the first curved shape of the endoscope insertion portion are simultaneously displayed on the monitor.

18. The endoscopy support system according to claim 2, wherein
the one or more processors are configured to be switchable between
a fourth display mode in which the first curved shape of the endoscope insertion portion, a plurality of the endoscopic images, and marks arranged on an insertion time line obtained by straightening the first curved shape of the endoscope insertion portion and indicating capturing timings of a plurality of the endoscopic images are displayed on the monitor and
a second display mode in which an endoscopic image selected by a user, a second curved shape of an endoscope insertion portion corresponding to the endoscopic image selected, and the first curved shape of the endoscope insertion portion are simultaneously displayed on the monitor.

19. An endoscopy support method comprising:
acquiring, in endoscopy, a first curved shape of an endoscope insertion portion when an endoscope distal end portion is located at a predetermined site, an endoscopic image captured in the endoscopy, and a second curved shape of an endoscope insertion portion when the endoscopic image is captured, and
displaying the first curved shape of the endoscope insertion portion and the second curved shape of the endoscope insertion portion simultaneously on a monitor.

20. A storage medium storing a program that causes a computer to perform:
a process of acquiring, in endoscopy, a first curved shape of an endoscope insertion portion when an endoscope distal end portion is located at a predetermined site, an endoscopic image captured in the endoscopy, and a second curved shape of an endoscope insertion portion when the endoscopic image is captured; and
a process of displaying the first curved shape of the endoscope insertion portion and the second curved shape of the endoscope insertion portion on simultaneously a monitor.
